(19) United States
(12) Patent Application Publication
(10) Pub. No.: US 2008/0254456 A1
(43) Pub. Date: Oct. 16, 2008

(54) GENE MARKER FOR HUMAN HEPATOCELLULAR CARCINOMA DIAGNOSIS

(76) Inventor: Jung-Yaw LIN, Taipei (TW)

Correspondence Address: BIRCH STEWART KOLASCH & BIRCH, PO BOX 747, FALLS CHURCH, VA, 22040-0747, US

(21) Appl. No.: 11/735,909

(22) Filed: Apr. 16, 2007

(51) Int. Cl. C12Q 1/68; C07H 21/04; C12M 3/00

(52) U.S. Cl. 435/6; 435/287.2; 536/24.3

(57) ABSTRACT

The present invention relates to a gene marker for diagnosis of human hepatocellular carcinoma (HCC), which is selected from the group consisting of IGF2, VEGF, MET, SUMO2, CDK4, MMP9, PLK1, AFP, Rb1, CYP3A4, LAP3, RIZ, DLC1, ITIH1, FetB, ALDOB, SULT2A1, ASS, HP, SERIND1, EI24, HGD, RODH, F2, SORD, and KNG. The HCC is diagnosed effectively and efficiently based on detecting the expression levels of the present gene marker from the liver tissue sample of an individual to be diagnosed.

### BACKGROUND OF THE INVENTION

1. Field of the Invention

The present invention relates to a gene marker for diagnosis of cancer, especially for diagnosis of human hepatocellular carcinoma (HCC).

2. The Prior Arts

Hepatocellular carcinoma (HCC) is a highly prevalent cancer disease worldwide. Hepatic B virus (HBV), hepatic C virus (HCV) and aflatoxin B1 are suggested to be the most important risk factors for hepatocarcinogenesis. During the development of HCC, normal liver cells would be transformed into highly malignant cells by a series of genetic aberrances. With different processes of genetic alternations, HCC may proceed into different stages of tumor, which can be classified by several indicators of clinicopathological status, such as differentiation grade, tumor size, number of tumor, and invasiveness. However, none of those classification methods could perfectly predict the prognosis of HCC in patients. Thus, it is important that identify specific markers that are critical for tumor initiation and tumor progression.

In the past decade, many studies involving in HCC have focused on chromosomal aberrations by comparative genomic hybridization (CGH) and microsatellite analysis, and frequent allelic loss of loci at chromosomes 1p, 4q, 6q, 8p, 13q, 16q and 17p. The CGH is used to investigate the increasing or decreasing of chromosome section in HCC tissue by chromosome hybridization and thereby identify oncogenes or cancer suppressor genes that might be involved in tumor initiation. Because this method has a limitation that can't focus on a single gene to processing analysis, it often identifies chromosome loci with 20-50 genes. Therefore, one could not affirm which gene in the chromosome loci will be expressed in the HCC tissue and identify change of gene expression causing by non-chromosomal aberrations.

Recently, the cDNA microarray and the serial analysis of gene expression (SAGE) technologies have been widely used to analyze the gene expression differences between tumor and normal tissues, and provide a new basis for identifying genes that are activated or suppressed in HCC. However, the technology has many problematic factors in gene expression profile data.

Due to the current limitation to identify gene markers that can perfectly predict the prognosis of HCC in patients, it is important to search and identify suitable gene markers in HCC by utilizing another method.

### SUMMARY OF THE INVENTION

In order to overcome the drawbacks of the prior art as described above, a primary object of the present invention is to provide gene markers for detecting human hepatocellular carcinoma (HCC) properly.

Another object of the present invention is to provide a method for detecting HCC.

To fulfill the objective of the present invention, gene markers for diagnosis of HCC are selected from 459 genes that are dysregulated in HCC tissues.

Compared with normal tissues, the gene markers of the present invention are either up-regulated or down-regulated in HCC tissues from 45 HCC patients. The mean expression levels of 96 gene markers were found to be 2-fold differentially expression between the tumor and normal tissues, with 19 overexpressed and 77 underexpressed.

In addition, a method for detecting HCC according to the present invention comprises the steps of:
(1) obtaining an liver tissue sample from an individual to be diagnosed;
(2) determining the expression levels of the abovementioned gene markers in the liver tissue sample;
(3) comparing the expression levels of the gene markers in the liver tissue sample of step (2) with the expression levels of the tumor markers in non-HCC liver tissues; and
(4) identifying if the individual being diagnosed is affected with the HCC or not from result of step (3).

The gene marker according to the present invention can be applied as a diagnostic tool in detecting HCC including early stage (I-II) and later stage (III-IV).

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### DETAILED DESCRIPTION OF THE PREFERRED EMBODIMENT

The gene marker of the present invention can be applied in diagnosis of HCC. Compared with normal liver tissues, the expression level of gene marker according to the present invention is either up-regulated or down-regulated in liver tissue of HCC patient. Following comparison between tumor tissues and corresponding normal tissues, the examples for up-regulated gene marker are MET, N-RAS, MMP9, etc. On the contrary, the examples for down-regulated gene marker are P73, TSLC, APC, etc. Functional classes of 96 differentially expressed gene markers included liver metabolism (20%), cytoskeleton or cell adhesion (8.4%), cell cycle or apoptosis (11.6%), tumor suppression (10.5%), signal transduction or oncogene (8.4%), protease/proteinase or its inhibitor (11.6%), tumor association (3.2%), and liver secretory or other function (26.3%).

The present invention also discloses 26 of 96 gene markers that can be applied in diagnosing late-stage (III-IV) HCC. The gene makers associated with developing of HCC in late-stage include IGF2, VEGF, MET, SUMO2, CDK4, MMP9, PLK1, AFP, Rb1, CYP3A4, LAP3, RIZ, DLC1, ITIH1, FetB, ALDOB, SULT2A1, ASS, HP, SERIND1, EI24, HGD, RODH, F2, SORD and KNG.

Histological analysis revealed that tumor size, infection with HBV or HCV, and differentiation grade were not correlated with portal vein invasion (PVI), which has been suggested to be the most important issue for survival in cancer. The present invention further discloses 16 of 26 gene markers that can be applied in diagnosing HCC with PVI. The 16 PVI-associated gene markers include SUMO2, RBI, MET, DLC1, VEGF, PLK1, CDK4, AFP, F2, FetB, RIZ, SULT2A1, RODH, LAP3, CYP3A4 and SERPIND1.

On the other hand, the present invention more further discloses that 7 of 16 PVI-associated gene markers are important in death. The poor prognosis gene marker associated with death rate include AFP, SULT2A1, RODH, RIZ, LAP3, F2, and VEGF, which are important risk factors in short-term survive.

The abovementioned differential (or change) of expression level of the gene markers in the HCC tissues according to the present invention can be easily determined with the relevant known gene analysis techniques, which include but are not limited to north blotting, real time PCR and so on, by the person skilled in the art by reading the disclosure of the specification.

The present invention also provides a method for detecting and identifying HCC since the expression levels of the gene markers in the liver tissues will be changed in accordance with the invasion of tumor cells. The method for detecting HCC according to the present invention first obtains a liver tissue sample from an individual to be diagnosed; and then analyzes the expression levels of the present gene markers in the liver tissue sample through the known methods. Then the expression levels of the gene markers in the liver tissue sample are compared with the expression levels of the gene markers in normal liver tissues. Lastly, comparison result of the expression levels of gene markers is used to determine whether the expression level has been changed (up-regulated or down-regulated), and to identify if the individual being diagnosed is affected with the HCC or not. The changed expression level should be more than 2-fold differentially when compare with normal liver tissues.

The abovementioned normal liver tissues could be obtained from other individual, who is not being affected by the HCC; or other parts of the liver tissue sample to be diagnosed, which is not invaded by the HCC, in the same individual.

The changes of expression level of the gene markers in the HCC tissues according to the present invention can be easily determined with the relevant known gene analysis techniques, which include but are not limited to north blotting, real time PCR and so on, by the person skilled in the art by reading the disclosure of the specification.

The abovementioned HCC comprises clinical stage I, II, III, and IV. In addition, the accuracy of diagnosis of HCC performed with the method of the present invention can be increased through combining the analysis results of the expression levels from multiple of the present gene marker.

One could perfectly predict if the individual being diagnosed is affected with the HCC, stage of HCC, prognosis of HCC in patients by suitable gene markers being disclosed in the present specification.

After reading the disclosure of the specification, the person skilled in the art could produce DNA biochips with the present gene marker by known technique and use the DNA biochips to diagnosis HCC and predict the development of late-stage HCC and prognosis of HCC in patients. On the other hand, one skilled in the art also could prepare protein biochips with transcription products of the gene marker of present invention by known technique and thereby develop inhibitors against the gene marker.

The person skilled in the art also could screen medicines for treating HCC by identifying which can activate or suppress expression of the present gene marker. For example, one could fuse a green fluorescence protein gene to promoter of the present gene marker and detect the fluorescence intensity in the cells of test animal after treated with the test medicine.

### EXAMPLE 1

We obtained informed consent from 45 patients with HCC who underwent hepatectomy in Taiwan University Hospital. Primary HCCs and adjacent normal liver tissues were obtained. Clinicopathological data were obtained from medical records and clinical diagnosis. Serology analysis revealed 33 cases infected with hepatitis B, 9 positive for HCV, 2 of 9 positive for both HBV and HCV and 1 without virus infection, Histopathological classification involved the Edmondson grading system; serum AFP level was detected by standard ELISA; vascular vein invasion was determined by histochemical analysis and clinical staging by the Vauthey system. No significant differences were found between HBV- and HCV-positive status with respect to grade of differentiation, vessel invasion, cirrhosis status, or tumor stage.

To identify the genes differentially expressed in HCC development, we used the known oligo-capping method to analyze the number of transcripts in tumor and normal tissues in full-length cDNA libraries by high-throughput DNA sequence analysis. Construction of full-length cDNA libraries involved the oligo-capping analysis of the copy number of genes in HCC and normal liver tissue, and whole transcripts were sequenced and underwent BLAST analysis with the GenBank non-redundant nucleotide library maintained by the National Center for Biotechnology Information (NCBI) (Ota, T. et al. Complete sequencing and characterization of 21,243 full-length human cDNAs. Nat. Genet. 36, 40-45 (2004).).

These sequencing results from paired HCC and liver samples were grouped into 52,629 known (p value<1×10⁻⁶) and 5,652 unknown cDNAs and further integrated into 3,513 and 2,692 genes for HCC tissues and nontumor adjacent liver tissues, respectively. A total of 180 candidate gene markers were identified to be overexpressed in HCC, with 279 genes underexpressed.

In the present invention, Real-Time Quantitative PCR (Q-PCR) and Statistical and Phylogenetic Analyses were also performed to analyze the gene expression profile of HCC tissue pairs. Total RNA was extracted from HCC and normal liver tissues adjacent to tumors with use of Trizol reagent (Invitrogen). The total RNA was quantitated, and first-strand cDNA was prepared by use of the SuperScript II cDNA Synthesis method (Invitrogen) with oligo-dT primers. The transcript levels were determined by quantification assay on real-time RT-PCR involving iCycler instruments (Biorad) and fluorescence resonance energy-transfer (FRET) hybridization methodology. The specific primer sets were designed by use of Beacon designer 4.0 (Premier Biosoft International). Any two Q-PCR C_{T} (Cycle of threshold) values had to be less than 1.0 for validating the reproducibility of data; otherwise, the data were removed and repeated. Corrections for sample differences involved normalization to the reference gene (housekeeping gene). The constitutively expressed gene glyceraldehyde-3-phosphate dehydrogenase (GAPDH) was taken as the internal control.

The mean expression levels of 96 genes were found to be 2-fold differentially expression between HCC and normal tissues, with 19 overexpressed (Table 1) and 77 underexpressed (Table 2). A high proportion was found to be down-regulated on chromosomal arms 4q, 8p, 10p, 11p, 13q, 16q, and 17p, with the up-regulated genes preferentially located on chromosomal 16p, 17q and 20q loci 2,3, suggesting that the genomic alternations should result in differential expression of genes.

TABLE 1
19 overexpressed genes in HCC

| | | | |
|---|---|---|---|
| ID | Symbol | Locus | Average fold (Percentage) |

Cytoskeleton/Cell adhesin

| | | | |
|---|---|---|---|
| NMGL8592 | ANAX2 | 15q21 | 3.1 (51%) |
| NMGL12375 | CAP1 | 1p34 | 2.5 (39%) |
| NMGL1866 | CFL1 | 11q13 | 2.5 (34%) |

Cell cycle/Apoptosis

| | | | |
|---|---|---|---|
| NMGL29967 | PLK1 | 16p12 | 11.2 (63%) |
| NMGL27576 | CDK2 | 12q13 | 10.9 (46%) |
| NMGL14169 | CCNE | 19q12 | 6.5 (63%) |
| NMGL16731 | CDK4 | 12q14 | 4.7 (51%) |

Signal transduction/Oncogene

| | | | |
|---|---|---|---|
| NMGL19056 | erbB2 | 17q21 | 6.8 (54%) |
| NMGL8898 | MET | 7q31 | 4.8 (68%) |
| NMGL19506 | N-RAS | 1p13 | 2.5 (49%) |

Protease/Protease inhibitor

| | | | |
|---|---|---|---|
| NMGL22239 | MMP9 | 20q11–13 | 4.6 (71%) |

Tumor-associated protein

| | | | |
|---|---|---|---|
| NMGL26424 | AFP | 4q11 | 8.8 (48%) |
| NMGL25914 | NME1 | 17q21–25 | 3.3 (53%) |
| NMGL15432 | CRA | 1q12 | 2.4 (38%) |

Secretory/Other

| | | | |
|---|---|---|---|
| NMGL10242 | IGF2 | 11p15 | 11.9 (69%) |
| NMGL29019 | EIF4A1 | 17p13 | 6.8 (32%) |
| NMGL18894 | SUMO2 | 17q21–25 | 4.0 (68%) |
| NMGL15924 | UBA52 | 19p13 | 3.1 (35%) |
| NMGL11379 | VEGF | 6p12 | 2.6 (49%) |

TABLE 2
77 underexpressed genes in HCC

| | | | |
|---|---|---|---|
| ID | Symbol | Locus | Average fold (Percentage) |

Liver function and metabolic enzymes

| | | | | |
|---|---|---|---|---|
| NMGL21762 | CYP3A4 | 7q21 | 117.5 | (78%) |
| NMGL3657 | ADH4 | 4q21–23 | 113.8 | (86%) |
| NMGL14874 | RODH | 12q13 | 55.3 | (67%) |
| NMGL5652 | SULT2A1 | 19q13 | 41.6 | (58%) |
| NMGL15705 | ALDOB | 9q21 | 35.4 | (73%) |
| NMGL13701 | AGXT | 2q36 | 33.4 | (53%) |
| NMGL25890 | LIPC | 15q21 | 17.0 | (58%) |
| NMGL2358 | ASS | 9q34 | 16.6 | (73%) |
| NMGL4245 | DIO1 | 1p33 | 15.2 | (58%) |
| NMGL4920 | TAT | 16q22 | 11.0 | (62%) |
| NMGL24762 | HGD | 3p21 | 10.3 | (71%) |
| NMGL2634 | SORD | 15q15 | 9.1 | (67%) |
| NMGL8499 | HMOX | 22q12 | 8.9 | (56%) |
| NMGL12 | ADH1B | 4q21–23 | 7.6 | (70%) |
| NMGL26676 | ADH6 | 4q21–23 | 5.6 | (62%) |
| NMGL14775 | HMGCL | 1p36 | 5.2 | (61%) |
| NMGL11508 | GSTA1 | 6p12 | 3.5 | (54%) |
| NMGL12516 | BHMT2 | 5q13 | 2.6 | (61%) |
| NMGL23151 | PEMT | 17p11–13 | 2.1 | (51%) |

Cytoskeleton/Cell adhesin

| | | | | |
|---|---|---|---|---|
| NMGL23334 | KAI1 | 11p11 | 14.1 | (41%) |
| NMGL19722 | TMSB10 | 2p11 | 12.6 | (44%) |
| NMGL3258 | CDH1 | 16q22 | 3.0 | (56%) |
| NMGL26829 | PFN1 | 17q13 | 2.2 | (34%) |
| NMGL9951 | VIM | 10p13 | 2.2 | (33%) |

Cell cycle/Apoptosis

| | | | | |
|---|---|---|---|---|
| NMGL15261 | FASTK | 7q35 | 8.9 | (36%) |
| NMGL7797 | CDKN2A | 9p21 | 3.7 | (49%) |
| NMGL11556 | CCND1 | 11q13 | 3.2 | (56%) |
| NMGL26970 | CDKN1B | 12p13 | 3.1 | (39%) |
| NMGL19047 | RTN4 | 2p16 | 2.2 | (60%) |
| NMGL3411 | CRI1 | 15q21 | 2.1 | (45%) |
| NMGL24825 | TNFSF10 | 2p26 | 2.0 | (39%) |

Tumor Suppressor

| | | | | |
|---|---|---|---|---|
| NMGL4296 | LZTS1 | 8p22 | 20.1 | (71%) |
| NMGL25584 | RB1 | 13q14 | 13.6 | (62%) |
| NMGL6396 | P73 | 1p36 | 11.6 | (65%) |
| NMGL1920 | SOCS1 | 16p13 | 7.3 | (56%) |
| NMGL12888 | DLC1 | 8p22 | 6.5 | (60%) |
| NMGL24924 | H-rev107 | 11p15 | 5.5 | (71%) |
| NMGL5469 | RIZ | 1p36 | 5.1 | (51%) |
| NMGL1530 | TSLC | 11q23 | 4.7 | (43%) |
| NMGL26796 | APC | 5q21 | 2.9 | (38%) |
| NMGL11907 | HIC1 | 17p13 | 2.7 | (63%) |

Signal transduction/Oncogene

| | | | | |
|---|---|---|---|---|
| NMGL15768 | RGS1 | 1q31 | 8.5 | (50%) |
| NMGL4458 | STAT1 | 2q32 | 3.5 | (33%) |
| NMGL11154 | SFN | 1p36 | 2.7 | (61%) |
| NMGL23331 | PTP4A1 | 6q12 | 2.7 | (31%) |
| NMGL11436 | Rac1 | 7p22 | 2.6 | (34%) |

Protease/Protease inhibitor

| | | | | |
|---|---|---|---|---|
| NMGL14238 | FETA | 3p26 | 81.9 | (62%) |
| NMGL23223 | KNG | 3p26 | 52.4 | (56%) |
| NMGL4434 | SERPIND1 | 22q11 | 17.6 | (66%) |
| NMGL771 | ITIH1 | 3p21 | 16.6 | (64%) |
| NMGL3219 | FetB | 3p26 | 16.4 | (65%) |
| NMGL28527 | SERPINF1 | 17p11–13 | 12.2 | (53%) |
| NMGL4494 | HRG | 3p26 | 10.2 | (63%) |
| NMGL15855 | ITIH2 | 10p15 | 7.2 | (35%) |
| NMGL14385 | SERPINE1 | 7q21 | 4.7 | (50%) |
| NMGL7737 | LAP3 | 4p15 | 2.6 | (50%) |

Secretory/Other

| | | | | |
|---|---|---|---|---|
| NMGL19590 | F2 | 11p11 | 117.6 | (78%) |
| NMGL11133 | HP | 16q22 | 83.9 | (80%) |
| NMGL8031 | AA4 | 11p15 | 56.2 | (70%) |
| NMGL7761 | MST1 | 3p21 | 22.4 | (51%) |
| NMGL2322 | FGB | 4q28 | 16.8 | (78%) |
| NMGL18765 | FGG | 4q28 | 13.5 | (69%) |
| NMGL4290 | F11 | 4q35 | 13.5 | (48%) |
| NMGL10308 | PLG | 6q26 | 12.8 | (79%) |
| NMGL5778 | CPB2 | 13q14 | 12.6 | (59%) |
| NMGL5703 | KLKB1 | 4q34 | 12.5 | (70%) |
| NMGL12234 | LBP | 20q11 | 12.0 | (44%) |
| NMGL3429 | FGL1 | 8p22 | 11.1 | (50%) |
| NMGL2709 | GPATC3 | 1p35 | 8.4 | (57%) |
| NMGL25923 | TM7SF3 | 12q11 | 6.5 | (59%) |
| NMGL20823 | HFL1 | 1q32 | 5.8 | (58%) |
| NMGL7713 | MBNL2 | 13q22 | 5.2 | (63%) |
| NMGL25065 | C1s | 12p13 | 4.3 | (44%) |
| NMGL6771 | VTN | 17q11 | 4.1 | (37%) |
| NMGL11601 | CD14 | 5q22–32 | 3.3 | (57%) |
| NMGL19104 | CD74 | 5q31 | 3.1 | (53%) |
| NMGL11733 | EI24 | 11q24 | 2.3 | (51%) |

Functional classes of 96 differentially expressed genes (more than 2-fold) included liver metabolism (20%), cytoskeleton or cell adhesion (8.4%), cell cycle or apoptosis (11.6%), tumor suppression (10.5%), signal transduction or oncogene (8.4%), protease/proteinase or its inhibitor (11.6%), tumor association (3.2%), and liver secretory or other function (26.3%). 19 of these 96 genes were shown to be 2.5 to 11.9-fold overexpression in HCC tissues with major categories including cytoskeleton, cell cycle, signal transduction or oncogene, protease, tumor-associated proteins, and secretory proteins or other functions (Table 1). Of the 77 genes 2.1 to 117.6-fold underexpressed in HCC, their functions were mainly involved in lever metabolism, cytoskeleton or cell adhesion, cell cycle or apoptosis, tumor suppression, signal transduction, protease inhibitor, and secretory or other functions (Table 2).

For further investigation of gene expression patterns associated with HCC progression, these HCC tissue pairs could be grouped into early stage I-II (n=22) and advanced stage III-IV (n=23) cancer according to portal vein invasion (PVI), tumor size and tumor number by Vauthey system. We performed Mann-Whitney statistic analysis to identify genes with significantly differential expression patterns between early and late-stage HCC pairs, and those genes were hierarchical clustered visualized by TreeView opensource software (http://jtreeview.sourceforge.net).

According to the results of the Mann-Whitney statistic analysis, we can found 26 of 96 gene markers associated with late-stage HCC and HCC progression. Interestingly, several genes involved in liver function were identified as late-stage HCC-related genes; RODH, CYP3A4, SULT2A1, and LAP3 were involved in metabolic processes, whereas FetB, F2, HFL1 and ITIH1 were secretory functional proteins.

Histological analysis revealed that tumor size, infection with HBV or HCV, and differentiation grade were not correlated with portal vein invasion (PVI), which has been suggested to be the most important issue for survival in cancer. In a short-term observation after resection, the median survival time of patients with or without PVI was 9 and 25 months, respectively, which may indicate the stronger relation between PVI and survival rate than tumor size and tumor differentiation, especially in short-term survival. Serum AFP level (>100 ng/ml or less), a well-accepted poor prognosis marker, was also associated with death rate. For further identification of the gene expression markers, we statistically analyzed features of pathologic development of tumors: tumor size, vascular invasion, and death. Interestingly, 16 of 23 PVI-associated genes were also those related to advanced-stage HCC, with less correlation among tumor size, differentiation grade and HCC stage and this 16 PVI-associated gene included SUMO2, RBI, MET, DLC1, VEGF, PLK1, CDK4, AFP, F2, FetB, RIZ, SULT2A1, RODH, LAP3, CYP3A4 and SERPIND1

We further applied Kaplan-Meier and Cox-regression analysis (SPSS software package, SPSS Inc.) to identify markers important in death and found 7 of 16 PVI-associated genes, including AFP, SULT2A1, RODH, RIZ, LAP3, F2, and VEGF, which suggest that these genes may be important risk factors in short-term survive.

1-14. (canceled)

15. A gene marker for human hepatocellular carcinoma (HCC) diagnosis, which is selected from the group consisting of IGF2, VEGF, MET, SUMO2, CDK4, MMP9, PLK1, AFP, Rb1, CYP3A4, LAP3, RIZ, DLC1, ITIH1, FetB, ALDOB, SULT2A1, ASS, HP, SERIND1, EI24, HGD, RODH, F2, SORD, and KNG.

16. A gene marker as claimed in claim 15, wherein the gene marker is up-regulated in liver tissues from HCC patients compared with normal liver tissues.

17. A gene marker as claimed in claim 16, wherein the gene marker is selected from the group consisting of MET, N-RAS, and MMP9.

18. A gene marker as claimed in claim 15, wherein the gene marker is down-regulated in liver tissues from HCC patients compared with normal liver tissues.

19. A gene marker as claimed in claim 18, wherein the gene marker is selected from the group consisting of P73, TSLC, and APC.

20. A gene marker for human hepatocellular carcinoma (HCC) diagnosis, which is associated with the HCC progression and is selected from the group consisting of IGF2, VEGF, MET, SUMO2, CDK4, MMP9, PLK1, AFP, Rb1, CYP3A4, LAP3, RIZ, DLC1, ITIH1, FetB, ALDOB, SULT2A1, ASS, HP, SERIND1, EI24, HGD, RODH, F2, SORD, and KNG.

21. A gene marker for human hepatocellular carcinoma (HCC) diagnosis, which is associated with the HCC progression and portal vein invasion (PVI) and is selected from the group consisting of SUMO2, RB1, MET, DLC1, VEGF, PLK1, CDK4, AFP, F2, FetB, RIZ, SULT2A1, RODH, LAP3, CYP3A4, and SERPIND1.

22. A gene marker for human hepatocellular carcinoma (HCC) diagnosis, which is associated with poor prognosis survival rate in HCC patients and is selected from the group consisting of AFP, SULT2A1, RODH, RIZ, LAP3, F2, and VEGF.

23. A DNA biochip for human hepatocellular carcinoma (HCC) diagnosis, which comprises a nucleotide fragment of the gene marker as claim in claim 15.

24. A DNA biochip for human hepatocellular carcinoma (HCC) diagnosis, which comprises a nucleotide fragment of the gene marker as claim in claim 20.

25. A DNA biochip for human hepatocellular carcinoma (HCC) diagnosis, which comprises a nucleotide fragment of the gene marker as claim in claim 21.

26. A DNA biochip for human hepatocellular carcinoma (HCC) diagnosis, which comprises a nucleotide fragment of the gene marker as claim in claim 22.

27. A protein biochip for human hepatocellular carcinoma (HCC) diagnosis, which comprises a transcription product of the gene marker as claim in claim 15.

28. A protein biochip for human hepatocellular carcinoma (HCC) diagnosis, which comprises a transcription product of the gene marker as claim in claim 20.

29. A protein biochip for human hepatocellular carcinoma (HCC) diagnosis, which comprises a transcription product of the gene marker as claim in claim 21.

30. A protein biochip for human hepatocellular carcinoma (HCC) diagnosis, which comprises a transcription product of the gene marker as claim in claim 22.

31. A method for detecting HCC, which comprises the steps of:
(1) obtaining a liver tissue sample from an individual to be diagnosed;
(2) determining expression levels of the gene marker as claimed in claim 15 in the ovarian tissue sample;
(3) comparing the expression levels of the gene marker in the liver tissue sample of step (2) with the expression levels of the gene marker in non-HCC tissue; and
(4) determining if the individual being diagnosed is affected with the HCC or not from the result of step (3).

32. A method as claimed in claim 31, wherein the expression levels of the gene marker is analyzed by north blotting or real time PCR.

33. A method as claimed in claim 31, wherein the non-HCC tissue is obtained from malignant cells non-invaded region of the same individual to be diagnosed.

34. A method as claimed in claim 31, wherein the HCC comprises clinical stage I, II, III and IV.
